(19) United States
(12) Patent Application Publication
(10) Pub. No.: US 2014/0000640 A1
(43) Pub. Date: Jan. 2, 2014

(54) LIGHT-HARDENING NAIL POLISH

(71) Applicant: Ralf Janda, Oestrich-Winkel (DE)

(72) Inventor: Ralf Janda, Oestrich-Winkel (DE)

(73) Assignee: WILDE COSMETICS GMHH, Oestrich-Winkel (DE)

(21) Appl. No.: 14/005,322

(22) PCT Filed: Mar. 15, 2012
(86) PCT No.: PCT/EP2012/001166

§ 371 Date: Sep. 18, 2013

(30) Foreign Application Priority Data
Mar. 16, 2011 (DE) 10 2011 014 149.9

(51) Int. Cl. A61K 8/37; A61Q 3/02; A45D 29/11

(52) U.S. Cl. CPC A61K 8/37; A45D 29/11; A61Q 3/02
USPC 132/200; 424/61

(57) ABSTRACT

The present invention relates to a nail polish, especially a light-hardening nail polish, for application to a nail, for example a finger nail, toe nail or the like, and also to a method of nail polishing using the nail polish according to the invention. The nail polish according to the invention allows nail polishing in simple manner and provides polish layers which are especially durable.

### BACKGROUND TO THE INVENTION

Hitherto nail polishes that consist of solutions of nitrocellulose in solvents (for example ethyl acetate, butyl acetate, methyl ethyl ketone or the like) and other additives (pigments, plasticisers, SiO₂ etc.) have been used in the main. After application of the polish to a nail, the solvent evaporates off and the nitrocellulose forms a polish film on the nail. That evaporation process lasts some minutes before the polish can be touched at all without being damaged. Afterwards it takes up to 15 minutes for the polish film to become “impact-resistant”.

The solvent for a nail polish of such a kind constitutes a safety problem especially in transportation. Also, it is an aim in line with the VOC Directive (EU Directive 1999/13/EC on the limitation of emissions of volatile organic compounds (VOC)) to reduce emissions of solvents or avoid them altogether. In some countries (for example, Switzerland) even, penalties have to be paid when solvent-containing products are imported.

In addition thereto, such solvent-containing nail polishes are not very stable or long-lasting after application. Depending on the stress conditions they are exposed to (dish-washing, hand-washing, sports, swimming, or other types of work using the hands) pieces of the polish layer very rapidly chip off, so that the polish often will already have become unsightly by the evening or the following day and have to be removed or renewed.

There are also nail polishes which can be hardened using light (usually UVA light). These polishes consist, for example, of mixtures of acrylates, methacrylates, pigments, photoinitiators, SiO₂ etc. Although these polishes fully harden rapidly when irradiated with light (for example, within 1 to 2 minutes), they do, however, have some disadvantages:

Before application, a large area of the finger nail has to be roughened, and a special bonding agent has to be applied first in order for the polish to adhere at all. As a rule, the bonding agent itself also has to be hardened using light before application of the polish.

After application of the polish and hardening thereof, a sealing agent must additionally be applied so that the polish acquires a glossy surface. That sealing agent also has to be hardened using light.

In addition, those multi-layer structures are frequently very brittle or also intrinsically unstable, so that they readily chip, for example if hit against an edge.

Also, those multi-layer structures cannot be easily removed using conventional nail polish removers, because the monomers used are crosslinkers, for example di- or tri-(meth)acrylates, and the resulting polymers are accordingly highly crosslinked and consequently brittle, and also insoluble in solvents. They have to be removed mechanically by filing, which, if done frequently, also results in the natural nail becoming ever thinner. Sometimes, small percentages of monofunctional acrylates/methacrylates are also added to the mixtures. If the fingers polished with such products are then bathed in acetone for 15 to 20 minutes, the soluble parts are dissolved away and the remaining polymer framework becomes so unstable that it can be prised off. Of course, the bathing in acetone for a number of minutes and repeated many times is not beneficial to the nail, the skin or to health overall.

There was accordingly a requirement for a solvent-free nail polish system which can be applied in simple manner, which adheres very well and is glossy, and which is easy to remove using conventional, even acetone-free, nail polish removers.

### BRIEF DESCRIPTION OF THE INVENTION

In order to solve that problem, the present invention provides a nail polish, especially a light-hardening nail polish, which comprises a photopolymerisable monofunctional acrylic acid ester monomer and a photoinitiator (acrylate system). The monofunctional acrylic acid ester monomer therein comprises at least one monomer selected from 2-[[(butylamino)carbonyl]oxy]-ethyl acrylate, ethoxylated (4) nonyl phenol acrylate and ethoxylated (8) nonyl phenol acrylate. The present invention furthermore provides a method of nail polishing wherein the nail polish, especially light-hardening nail polish, according to the invention is applied to a nail and caused to polymerise using light.

Preferred embodiments form the subject-matter of the dependent patent claims.

### DETAILED DESCRIPTION OF THE INVENTION

The inventor of the present invention has found that a nail polish that solves the problem of the invention must be present in liquid form for application, the liquid components having to be rapidly polymerisable, and no—or only small amounts of—crosslinkers being allowed to be present. In order to achieve rapid polymerisation, light hardening has been found to be especially advantageous. Customary monomers which are polymerisable by light hardening are, for example, monofunctional acrylates and methacrylates, for example methyl methacrylate, ethyl methacrylate, butyl methacrylate, isobornyl methacrylate, tetrahydrofurfuryl methacrylate and the like.

The extensive tests by the inventor have shown, however, that those customary monomers are not in principle suitable for such a nail polish, especially also because they have a very marked odour and so are extremely unpleasant for the user. Also, those customary monomers, if they are crosslinked using a photoinitiator, cannot as a rule be polymerised into films because inhibition resulting from atmospheric oxygen is so marked that the thin-layered products do not harden at all or only very incompletely.

This problem can be solved, for example, by dissolving further components, for example plasticisers, film-forming agents or the like, in those monomers, in order to improve their film-forming properties during polymerisation. Suitable further components are, for example, thermoplastic, that is to say non-crosslinked, polymers (for example polymethyl methacrylate, polystyrene, polyvinyl acetate, polyvinyl butyral or the like), sucrose benzoate or the like.

Although an improvement in film formation is in principle possible in that manner, the following further problems were recognised by the inventor:

1. Thermoplastic polymers, for example polymethyl methacrylate, polystyrene, polyvinyl acetate, polyvinyl butyral or the like, can be dissolved in customary monomers only in small amounts (up to about 20%), because the resulting solution thickens very quickly and becomes extremely viscous so that it becomes impossible to work with. For example, a monomer, for example polymethyl methacrylate, as a rule always still comprises residual amounts of initiator, for example dibenzoyl peroxide, originating from its synthesis, so that the solutions do not have adequate storage stability even at room temperature.

2. When such systems comprising customary monomers, for example methyl methacrylate, ethyl methacrylate, butyl methacrylate, isobornyl methacrylate, tetrahydrofurfuryl methacrylate or the like, are used with the relevant dissolved polymers, very brittle products are obtained after full hardening, which very rapidly splinter or chip off. Also, it is not possible to achieve adequate surface gloss.

It has been found by the inventor in surprising manner that sucrose benzoate can be dissolved in very large amounts (in some cases up to 80% by weight) in all the customary monomers the come into consideration. Especially when the sucrose benzoate content has been optimised, such mixtures, including added photoinitiator, can be applied to the natural nail in desired layer thicknesses and polymerised into high-gloss films. As both the non-crosslinked polymers and also, especially, sucrose benzoate are very readily soluble in many solvents, the polish layers can be removed again in simple manner using customary, even acetone-free, nail polish removers.

In this context it must be emphasised very especially that such products can also be removed using so-called solvent-free nail polish removers based on mixtures of dibasic esters of glutaric acid, adipic acid and succinic acid (dimethyl glutarates, dimethyl adipate, dimethyl succinate). Those esters, which do not count as solvents, are especially compatible with the skin and are employed in skin cleansers used to remove oils and other greasy constituents from the skin.

However, the inventor has also found that the polish films produced from those systems of customary monomers and sucrose benzoate are extremely brittle and fragile so that their use as a nail polish is not possible. In order to reduce the brittleness, it is in principle possible to add various plasticisers (for example, based on phthalate or citrate) in such concentrations as to achieve adequate elasticity of the hardened film. Unfortunately, however, this approach also proved unsuitable because the large amounts of plasticiser required (up to about 5%) caused the intrinsic strength of the material to be reduced to such an extent that even though it is no longer brittle in the “unstressed” state—that is to say it does not immediately splinter off when worn on the finger nail—it already flakes off after 1 to 2 days.

The inventor has now found that a nail polish according to the invention that is suitable for solving the problem must comprise a monofunctional monomer which polymerises into a highly elastic polymer resistant to breaking. At the same time, that monofunctional monomer should result in films which have a high gloss. Furthermore, sucrose benzoate should be soluble in the monomer in large amounts, and the inherent brittleness intrinsic to the sucrose benzoate removed by the monomer. The monomer, together with a suitable photoinitiator, results in a photopolymerisable acrylate system.

The inventor has found in surprising manner that the monofunctional monomers 2-[[(butylamino)carbonyl]oxy]ethyl acrylate (or 2-(((butylamino)carbonyl)oxy)ethyl ester; CAS No. 63225-53-6; formula 1), ethoxylated (4) nonyl phenol acrylate (CAS No. 50974-47-5; formula 2) and ethoxylated (8) nonyl phenol acrylate (CAS No. 50974-47-5; formula 3) have all the desired properties:
high dissolution power for sucrose benzoate and also for thermoplastic polymers such as polyvinyl acetate or polyvinyl butyral
maximum elasticity and resistance to breaking for the polymerised product
very good film-forming properties
very good gloss for the polymerised films
use of plasticisers unnecessary.

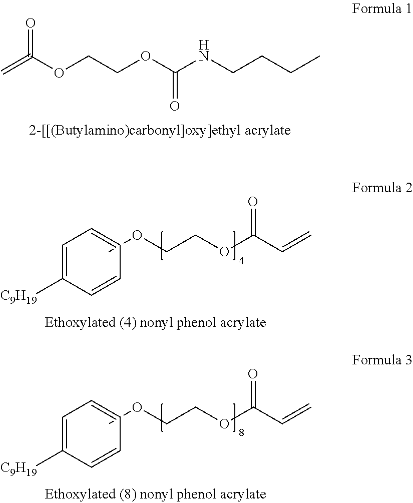

Sucrose benzoate denotes a molecule of sucrose (saccharose) which is esterified, preferably completely, with benzoic acid (CAS No. 12738-64-6; formula 4).

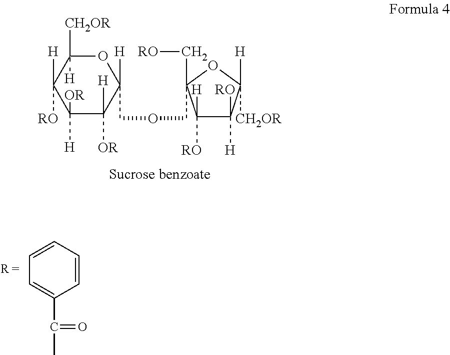

In general, acrylate/methacrylate systems, irrespective of whether they are crosslinked or not, exhibit a more or less thick inhibition layer on their surfaces which is caused by atmospheric oxygen. Surprisingly, the polymer preparations according to the invention do not exhibit this inhibition layer. The surface is scarcely even tacky. If these surfaces are polished with an oil-soaked absorbent wipe using light pressure, a lasting high gloss is very rapidly produced. Oils that come into consideration are preferably the oils used frequently in cosmetology: isopropyl myristate, castor oil, jojoba oil, almond oil and sunflower oil and also mixtures of those oils, because these even have skin-care properties.

The monomer 2-[[(butylamino)carbonyl]oxy]ethyl acrylate can be purchased, for example, under the tradename “Genomer 1122” from the company Rahn AG, Mirich, Switzerland.

The monomers ethoxylated (4) nonyl phenol acrylate and ethoxylated (8) nonyl phenol acrylate can be purchased, for example, under the tradenames Miramer M164 and Miramer M166 from the company Miwon Ltd., Kyonggi, Korea.

These monomers may be used as alternatives to one another or in admixture.

The nail polish, especially light-hardening nail polish, according to the invention comprises a photopolymerisable acrylate system of a monofunctional acrylic acid ester monomer and a photoinitiator. As photoinitiator there may be used any customary photoinitiator which is suitable for initiating a polymerisation reaction of an acrylate system.

Preferred examples of suitable photoinitiators are:
Darocur TPO, 2,4,6-trimethylbenzoyldiphenylphosphine oxide;
Darocur MBF, methyl phenyl glyoxylate;
Irgacure 184, 1-hydroxy-cyclohexyl-phenyl-ketone; and
Lucirin TPO-L, ethyl (2,4,6-trimethylbenzoyl) phenylphosphinate.

All those photoinitiators are obtainable from the company Ciba Spezialitätenchemie, now BASF AG, D-68623 Lampertheim, Germany.

A light-hardening nail polish as understood by this invention comprises a photopolymerisable acrylate system which comprises or consists of a monofunctional acrylic acid ester monomer and a photoinitiator. In this context the terms “light-hardening” and “photopolymerisable” are used to mean that the photoinitiator contained in the described systems can be caused to react by irradiation with light (electromagnetic radiation), especially light in the UV range having a wavelength of from 340 to 430 nm (UVA light), as a result of which a polymerisation reaction (chain-forming reaction) of the monofunctional acrylic acid ester monomers is initiated, which results in the formation of a polymer made from monomer units. In this context the polymerisation of the acrylate/methacrylate monomers is accomplished by free-radical polymerisation.

The nail polish according to the invention preferably comprises from 30 to 99% (if not otherwise indicated, all units are % by weight) monomer and from 1 to 10% photoinitiator, especially preferably from 39 to 85% monomer and 1 to 7% photoinitiator.

Preferably, mixtures of the monomer and sucrose benzoate and/or a thermoplastic polymer such as, especially preferably, polyvinyl acetate and/or polyvinyl butyral may be produced. Special preference is given to a polyvinyl acetate having a molar weight of from 10,000 to 100,000. Special preference is given to a polyvinyl butyral having a molar weight of from 50,000 to 120,000. By this means, viscosity, elasticity and surface hardness can be controlled and advantageously improved. The molar weight, or molar mass, of the polymers can be measured, for example, by Size Exclusion Chromatography (SEC) with a Low-Angle Laser Light Scattering (LALLS) standard detector.

The thermoplastic polyvinyl butyrals used (CAS No. 27360-07-2) are taken, for example, from the Butvar product group of the company Solutia Europe, Belgium. Preferably, the products B-90, molar mass 70,000-100,000, B-79, molar mass 50,000-80,000, B-98, molar mass 40,000-70,000 and B-76, molar mass 90,000-120,000 are used. The elongation capacities of these products are given as values from 100 to 110% and the glass transition temperatures as 62 to 78° C. The elongation capacity of the polymers can be determined, for example, by a standard tensile test, and the glass transition temperature can be determined, for example, by Differential Scanning calorimetry (DSC), for example in accordance with DIN 53765/ISO 11357-5.

The thermoplastic polyvinyl acetates used (CAS No. 9003-20-7) are taken, for example, from the Vinnapas product group of the company Wacker AG, Germany. Preferably, the products B 1.5 sp, molar mass 10,000-15,000, B 3 sp, molar mass 15,000-20,000, B 5 sp, molar mass 20,000-25,000, B 14 sp, molar mass 25,000-33,000, B 17 sp, molar mass 35,000-45,000, B 30 sp 45,000-55,000, molar mass B 60 sp, molar mass 55,000-70,000 and B 100, molar mass 80,000-100,000 are used. The glass transition temperatures of those products are given as 33 to 42° C. The glass transition temperature can be determined, for example, by Differential Scanning calorimetry (DSC), for example in accordance with DIN 53765/ISO 11357-5.

The nail polish according to the invention comprises preferably from 10 to 70%, more preferably from 30 to 45%, and especially from 34 to 41% sucrose benzoate, and/or from 2 to 20%, more preferably from 1 to 11%, and especially preferably from 1 to 5% of a thermoplastic polymer.

It is also possible to control the surface hardness by adding methacrylate monomers to the nail polish according to the invention. For example, isobornyl methacrylate, 2-hydroxyethyl methacrylate (HEMA; CAS No. 868-77-9), 3-hydroxypropyl methacrylate (HPMA; CAS No. 27813-02-1) or tetrahydrofurfuryl methacrylate can be added in amounts of up to 15% without brittleness that is too high for the intended application occurring as a result. Overall, the nail polish according to the invention comprises preferably from 30 to 99% acrylate/methacrylate monomer, especially preferably from 39 to 85% monomer.

Preferably, it is also possible to add small amounts of crosslinking monomers to the preparations. Diacrylates and/or dimethacrylates are especially preferred. These can be added in an amount of up to 25% without crucially affecting the good properties of dissolution in nail polish removers. By this means, gloss and abrasion resistance of the films can, if necessary, be improved.

Especially preferred diacrylates and dimethacrylates are:
Bis-GMA=2,2-bis[4(3′-methacryloyl-oxy-2′-hydroxy)propoxyphenyl]propane (CAS No. 1565-94-2);
UDMA=7,7,9-trimethyl-4,13-dioxo-3,14-dioxa-5,12-diazahexadecane-1,16-dioxy-dimethacrylate (CAS No. 41137-60-4);
UDA=7,7,9-trimethyl-4,13-dioxo-3,14-dioxa-5,12-diazahexadecane-1,16-dioxy-diacrylate;
Bis-EDMA=2,2-bis[4(3′-methacryloyl-oxy)ethoxyphenyl)]propane; and
TEGDMA=triethylene glycol dimethacrylate.

Preferably, in addition adhesion-imparting agents can be added. These can be added preferably in an amount of up to 10%, especially preferably in an amount of up to 5%.

Preferred examples of adhesion-imparting agents are:
PMGDM=bis(glyceryl dimethacrylate)pyromellitate (CAS No. 148019-46-9);
4-Meta=4-methacryloyl-oxypropyl-trimellitic anhydride;
MDP=10-methacryloyl-oxy-decyl dihydrogen phosphate;
2-methacryloyl-oxy-ethylphenyl phosphate;
ethylene glycol methacrylate phosphate; and
bis(2-methacryloyloxy)ethyl phosphate.

The nail polish according to the invention can preferably be applied to a nail and then caused to polymerise using light. Especially preferably, the polymerisation is carried out using light having a wavelength of from 340 to 430 nm. For that purpose there can be used, for example, a customary polymerisation light apparatus having, as light source, UV fluorescent tubes whose emission maximum is at about 370 nm or an LED apparatus having an emission maximum at about 400 nm. Application of the solvent-free nail polish system is simple and can be accomplished in one layer, for example using a brush. The nail polish adheres very well to the nail, both before and after polymerisation using light, preferably UV light in the wavelength range from 360 to 420 nm. The polymerised polish layers are glossy and can be removed again by simple means using conventional, even acetone-free, nail polish removers.

It has been found, especially, that the durability of the nail polish according to the invention, after application and polymerisation, is about 5 to 10 times greater, depending on the particular stress conditions, compared to conventional solvent-based nail polishes. Application of a nail polish according to the invention accordingly has to be renewed 5 to 10 times less frequently.

In addition, the nail polish according to the invention has immediate strength as a result of the light hardening. The polish layers obtained are distinguished by very good capacity for removal by dissolution.

Preferably, the nail polish of the invention can also comprise further additives, for example a colourant and/or a filler.

Preferred colourants are dyes, for example food dyes. Preferred examples are, amongst others:
Sicovit Amaranth 85E123, CI-16185;
Sicovit Azorubine 85E122, CI-14720;
Sicovit Cochineal Red 70E124, CI-16255; and
Sicovit Tartrazine 85E102, CI-19140,

all of which are obtainable from BASF AG.

Further preferred colourants are pigments. Preferred examples are, amongst others:
Sicovit Red (iron oxide red), CI-77491;
Sicovit Yellow (iron oxide yellow), CI-77492;
Sicovit Black (iron oxide black), CI-77499; and
Sicovit White (titanium dioxide), CI-77891,

all of which are obtainable from BASF AG.

Further examples of preferred pigments are:
Unipure Yellow LC 181 CI-77492;
Unipure Yellow LC 124 CI-47005:1;
Unipure Red LC 380 CI-77491;
Unipure Black LC 989 CI-77499;
Unipure Violet LC 526 CI-77742;
Unipure Blue LC 680 CI-77007;
Unipure Green LC 788 CI-77288;
Unipure Red LC 303 CI-15850;
Unipure Red LC 321 CI-45380:3;
Unipure Red LC 327 CI-45410:2; and
Unipure Blue LC 621 CI-42090:2,

all of which are obtainable from the company Sensient Cosmetic Technologies LCW, France.

Preferred fillers are, for example, pyrogenic silicic acids (silica), as are obtainable, for example, under the tradename “Aerosil” from the company EVONIK Industries.

In order to improve colour stability on fully hardening, the nail polish of the invention can also comprise further additives, for example an optical brightener or fluorescent whitening agent, for example 2,5-thiophenediylbis(5-tert-butyl-1,3-benzoxazole) (formula 5).

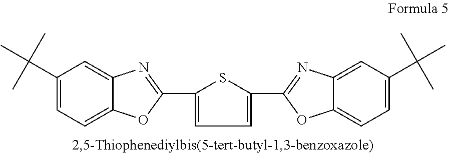 [SMILES: CC(C)(C)c1ccc2oc(-c3ccc(-c4nc5cc(C(C)(C)C)ccc5o4)s3)nc2c1]

2,5-Thiophenediylbis(5-tert-butyl-1,3-benzoxazole) is obtainable, for example, under the tradename Tinopal OB from the company Ciba Spezialitätenchemie, Germany.

A fluorescent whitening agent advantageously ensures constancy of colour on full hardening in various polymerisation light apparatuses. For example, full hardening can be carried out in a UV fluorescent tube apparatus having tubes whose emission maximum is at about 370 nm, as is most frequently used in the market currently, or in an LED apparatus whose emission maximum is at about 400 nm, currently a very modern version of apparatus. The different types of light of those apparatuses can result in different excitation of the electrons of the photoinitiator, which can, especially in the case of full hardening in the LED apparatus, result in initially very marked yellowish discolouration of the plastics material (ΔE≈5). Although this yellow discolouration is usually reversible and as a rule reverses itself after about 3 hours, it does detract from the initial impression of the colour of the polymerised product and, therefore, customer satisfaction. The yellow discolouration after full hardening in the LED apparatus can advantageously be prevented from the outset by addition of a suitable fluorescent whitening agent. A suitable fluorescent whitening agent is, for example, 2,5-thiophenediylbis(5-tert-butyl-1,3-benzoxazole) shown in Formula 5.

The mode of action of this fluorescent whitening agent is probably based on UV portions of the irradiated light being absorbed and the electrons of the molecule reaching higher energy states. When the excited state subsides, visible light is then emitted and the object appears whiter (brighter).

It has been found that an addition of 0.2% or less of 2,5-thiophenediylbis(5-tert-butyl-1,3-benzoxazole) was able to completely prevent the yellow tinge produced on polymerisation in the LED light apparatus. However, such large amounts may not be usable in practice as these may simultaneously cause other problems:
On the one hand, full hardening in the UV fluorescent tube apparatus can be adversely affected or prevented in relatively deep layers of material. At the surface, full hardening and therefore polymerisation shrinkage proceed significantly faster than deeper down, which can result in the formation of so-called “orange peel” at the surface. The fact that this effect does not occur, or occurs less, in the LED light apparatus could be due to the emitted light energy of this apparatus being markedly greater than that of the UV fluorescent tube apparatus so that the depth of irradiation into the material is also markedly greater. As a result, in the LED light apparatus, polymerisation proceeds in all layers uniformly.
On the other hand, certain colours (colour additives) can be adversely affected by excessive amounts of 2,5-thiophenediylbis(5-tert-butyl-1,3-benzoxazole) because this substance, in high concentrations, produces a marked bluish lustre. The amount of 2,5-thiophenediylbis(5-tert-butyl-1,3-benzoxazole) therefore has to be reduced until the afore-mentioned adverse effects can no longer be seen. It has been found that as a rule 0.10% or less of this fluorescent whitening agent is sufficient to reduce the yellow tinge produced in the LED light apparatus to the extent that it was no longer visually perceptible.

The nail polish according to the invention comprises preferably 0.10% or less of a fluorescent whitening agent, more preferably 0.05% or less, more preferably 0.0005 to 0.01%, and especially preferably 0.0008 to 0.005%. Very special preference is given to the nail polish according to the invention comprising 0.001% 2,5-thiophenediylbis(5-tert-butyl-1,3-benzoxazole).

### EXAMPLES

A number of examples of a nail polish according to the invention are set out hereinbelow.

All percentages therein denote percentages by weight, based on the complete composition. In all the formulations, Lucirin TPO-L (ethyl (2,4,6-trimethylbenzoyl) phenylphosphinate) from BASF AG was used as photoinitiator.

### Example 1

Formulation

| | |
|---|---|
| 40% | sucrose benzoate |
| 52% | Genomer 1122 |
| 3% | very finely divided SiO₂ (Aerosil) |
| 5% | photoinitiator |

### Example 2

Formulation

| | |
|---|---|
| 40% | sucrose benzoate |
| 50% | Genomer 1122 |
| 5% | polyvinyl acetate |
| 5% | photoinitiator |

### Example 3

Formulation

| | |
|---|---|
| 40% | sucrose benzoate |
| 39% | Genomer 1122 |
| 3% | very finely divided SiO₂ (Aerosil) |
| 3% | PMGDM |
| 5% | photoinitiator |

### Example 4

Formulation

| | |
|---|---|
| 38% | sucrose benzoate |
| 55% | Genomer 1122 |
| 2% | polyvinyl butyral |
| 5% | photoinitiator |

### Example 5

Formulation

| | |
|---|---|
| 70% | Genomer 1122 |
| 20% | polyvinyl acetate |
| 5% | PMGDM |
| 5% | photoinitiator |

### Example 6

Formulation

| | |
|---|---|
| 85% | Genomer 1122 |
| 10% | polyvinyl butyral |
| 5% | photoinitiator |

### Example 7

Formulation

| | |
|---|---|
| 40% | sucrose benzoate |
| 40% | Genomer 1122 |
| 10% | Miramer M166 |
| 5% | polyvinyl acetate |
| 5% | photoinitiator |

### Example 8

Formulation

| | |
|---|---|
| 40% | sucrose benzoate |
| 40% | Genomer 1122 |
| 15% | UDMA |
| 5% | photoinitiator |

### Example 9

Formulation

| | |
|---|---|
| 40% | sucrose benzoate |
| 38% | Miramer M164 |
| 8% | HEMA |
| 2% | UDMA |
| 3% | Bis-GMA |
| 2% | very finely divided SiO₂ (Aerosil) |
| 3% | PMGDM |
| 4% | photoinitiator |

### Example 10

Formulation

| | |
|---|---|
| 40% | sucrose benzoate |
| 32% | Miramer M166 |
| 10% | HEMA |
| 6% | UDMA |
| 3% | Bis-GMA |
| 2% | very finely divided SiO₂ (Aerosil) |
| 3% | PMGDM |
| 4% | photoinitiator |

### Example 11

Formulation

| | |
|---|---|
| 40% | sucrose benzoate |
| 37% | Miramer M164 |
| 8% | HEMA |
| 3% | very finely divided SiO₂ (Aerosil) |
| 5% | PMGDM |
| 7% | photoinitiator |

### Example 12

Formulation

| | |
|---|---|
| 35% | sucrose benzoate |
| 41% | Miramer M164 |
| 10% | HEMA |
| 8% | polyvinyl butyral |
| 2% | very finely divided SiO₂ (Aerosil) |
| 4% | photoinitiator |

### Example 13

Formulation

| | |
|---|---|
| 35% | sucrose benzoate |
| 41% | Miramer M164 |
| 10% | HPMA |
| 8% | polyvinyl acetate |
| 2% | very finely divided SiO₂ (Aerosil) |
| 4% | photoinitiator |

### Example 14

Formulation

| | |
|---|---|
| 35% | sucrose benzoate |
| 31% | Miramer M164 |
| 10% | HPMA |
| 7% | UDMA |
| 8% | polyvinyl acetate |
| 2% | very finely divided SiO₂ (Aerosil) |
| 3% | PMGDM |
| 4% | photoinitiator |

### Example 15

Formulation

| | |
|---|---|
| 34.999% | sucrose benzoate |
| 31% | Miramer M164 |
| 10% | HPMA |
| 7% | UDMA |
| 8% | polyvinyl acetate |
| 2% | very finely divided SiO₂ (Aerosil) |
| 3% | PMGDM |
| 4% | photoinitiator |
| 0.001% | Tinopal OB |

The formulations of all the Examples are liquid and can be easily applied to a nail, where they adhere well. After polymerisation using light in the wavelength range from 350 to 420 nm glossy polish layers are obtained, which adhere to the nail very well and are especially impact-resistant. The polish layers obtained can be easily removed again using conventional, even acetone-free, nail polish removers.

### Example 16

Testing of Durability:

The durability of the nail polishes according to the invention produced in Examples 1 to 8 was determined by the practical test described hereinbelow.

For that purpose, a questionnaire having the following criteria was developed:
Liftings=lifting-off at the edges of the nail polish coating.
Defects=losses of material at any location in the nail polish coating.
Loss of gloss=surface quality after being worn for the relevant duration.
Loss=total loss of the nail polish coating.

For each version of material, the practical test was carried out using 15 test subjects in such a manner that the nails of one hand were coated with one version and those of the other hand with another version. It was accordingly possible to test two versions of material in one test subject group at the same time.

The observation period was 1, 3 and 5 days.

The results are shown in Table 1, the number of defects stated always referring to individual finger nails. For example, 7 observations in the “Lifting” category means that, for the material in question, lifting-off at the edges was observed in the case of 7 coated finger nails. Versions of material which produced similar results were combined in one results group.

A conventional solvent-based nail polish applied in a comparison test already showed defects after one day, depending on the stress conditions it was exposed to.

TABLE 1

| Criteria | Liftings | | | Defects | | | Loss of gloss | | | Loss | | |
|---|---|---|---|---|---|---|---|---|---|---|---|---|
| Observation | 1 | 3 | 5 | 1 | 3 | 5 | 1 | 3 | 5 | 1 | 3 | 5 |
| period [days] | | | | | | | | | | | | |
| Example (No.) | 1, 2, 4, 7 | | | | | | | | | | | |
| Observations | 0-1 | 8-10 | 10-12 | 0 | 1-4 | 6-8 | 0 | 0 | 4-5 | 0 | 0 | 0 |
| Example (No.) | 3 | | | | | | | | | | | |
| Observations | 0 | 0 | 1-2 | 0 | 0 | 2-3 | 0 | 0 | 3-4 | 0 | 0 | 0 |
| Example (No.) | 5, 6 | | | | | | | | | | | |
| Observations | 4-8 | 9-12 | 15 | 2-3 | 4-5 | 9-12 | 0 | 4-6 | 7-10 | 0 | 0 | 1 |
| Example (No.) | 8 | | | | | | | | | | | |
| Observations | 0-1 | 4-5 | 6-9 | 0 | 0 | 0 | 0 | 0 | 1-3 | 0 | 0 | 0 |

### Example 17

Testing of Capacity for Removal by Dissolution Using Acetone-Free Nail Polish Removers:

For removal of the nail polish coating according to the invention from the finger nail by dissolution, a commercially available acetone-free nail polish remover was used. The composition of the commercially available nail polish remover used is as follows: 60% ethyl acetate, 37% isopropyl alcohol, 2% butylene glycol, 1% Ricinus Communis (Castor) Seed Oil.

The nail polish of the particular Example was applied to a specimen slide using a brush in a layer thickness (about 0.1 mm) and area (about 1×1 cm) applicable to its use. An absorbent wipe was soaked in the nail polish remover and laid for about 10 seconds on the polish layer. Then, in similar manner to actual removal of a nail polish from the finger, the absorbent wipe soaked in nail polish remover was passed over the polish layer using pressure and rubbing movements. The period until all of the nail polish layer had been removed from the specimen slide was timed.

The results are summarised in Table 2.

TABLE 2

Example number of the material compositions

| | 1 | 2 | 3 | 4 | 5 | 6 | 7 | 8 |
|---|---|---|---|---|---|---|---|---|
| Time [s] | 5 | 10 | 5 | 10 | 15 | 15 | 10 | 20 |

1-13. (canceled)

14: A light-hardening nail polish comprising
(a) a photopolymerizable monofunctional acrylic acid ester monomer and a photoinitiator, the monofunctional acrylic acid ester monomer comprising at least one monomer selected from 2-[[(butylamino)carbonyl]oxy]ethyl acrylate, ethoxylated (4) nonyl phenol acrylate and ethoxylated (8) nonyl phenol acrylate and mixtures thereof; and
(b) a thermoplastic polymer, sucrose benzoate, or a combination thereof, wherein the thermoplastic polymer is selected from polyvinyl acetate and polyvinyl butyral and mixtures thereof.

15: The light-hardening nail polish of claim 14, wherein the photoinitiator is selected from 2,4,6-trimethylbenzoyldiphenylphosphine oxide, methyl phenyl glyoxylate, 1-hydroxy-cyclohexyl-phenyl-ketone and ethyl (2,4,6-trimethylbenzoyl)phenylphosphinate.

16: The light-hardening nail polish of claim 14, further comprising a crosslinking agent, an adhesion-imparting agent, a colorant, a filler, an optical brightener, a fluorescent whitening agent, at least one methacrylate monomer, or combinations thereof.

17: The light-hardening nail polish of claim 16, wherein the crosslinking agent is a crosslinking monomer.

18: The light-hardening nail polish of claim 17, wherein the crosslinking monomer is a diacrylate monomer, a dimethacrylate monomer, or a combination thereof.

19: The light-hardening nail polish of claim 18, wherein the crosslinking monomer is selected from 2,2-bis[4(3′-methacryloyl-oxy-2′-hydroxy)propoxyphenyl]propane, 7,7,9-trimethyl-4,13-dioxo-3,14-dioxa-5,12-diazahexadecane-1,16-dioxy-dimethacrylate, 7,7,9-trimethyl-4,13-dioxo-3,14-dioxa-5,12-diazahexadecane-1,16-dioxy-diacrylate, 2,2-bis[4(3′-methacryloyl-oxy)ethoxyphenyl)]propane, triethylene glycol dimethacrylate and mixtures thereof.

20: The light-hardening nail polish of claim 16, wherein the adhesion-imparting agent is selected from bis(glyceryl dimethacrylate)pyromellitate, 4-methacryloyl-oxypropyl-trimellitic anhydride, 10-methacryloyl-oxy-decyl dihydrogen phosphate, 2-methacryloyl-oxy-ethylphenyl phosphate, ethylene glycol methacrylate phosphate, bis(2-methacryloyloxy)ethyl phosphate and mixtures thereof.

21: The light-hardening nail polish of claim 16, wherein the at least one methacrylate monomer is selected from isobornyl methacrylate, 2-hydroxyethyl methacrylate, 3-hydroxypropyl methacrylate and tetrahydrofurfuryl methacrylate and mixtures thereof.

22: A method for polishing a nail comprising applying a light-hardening nail polish of claim 14 to a nail and exposing the nail to light to polymerize the polish.

23: A method for polishing a nail comprising applying a light-hardening nail polish of claim 16 to a nail and exposing the nail to light to polymerize the polish.

24: The method of claim 22, wherein the light has a wavelength of from 340 to 430 nm.

25: The method of claim 23, wherein the light has a wavelength of from 340 to 430 nm.
